(19) United States
(12) Patent Application Publication
(10) Pub. No.: US 2020/0096480 A1
(43) Pub. Date: Mar. 26, 2020

(54) METHOD FOR ACQUIRING AND STORING BIOMETRIC DATA, IN PARTICULAR DATA OF BIOLOGICAL SAMPLES, METHOD FOR CONTROLLING A BIOLOGICAL SYSTEM, APPARATUS FOR CARRYING OUT THE METHODS, AND USES

(71) Applicant: Dietrich REICHWEIN, Berlin (DE)

(72) Inventor: Dietrich REICHWEIN, Berlin (DE)

(21) Appl. No.: 16/269,085

(22) Filed: Feb. 6, 2019

Related U.S. Application Data
(60) Provisional application No. 62/739,769, filed on Oct. 1, 2018.

(30) Foreign Application Priority Data
Sep. 21, 2018 (DE) 10 2018 216 095.3

(51) Int. Cl. G01N 27/90; G01N 33/483

(52) U.S. Cl. CPC G01N 27/9046; G01N 33/4833; G01N 27/9073

(57) ABSTRACT

A method for acquiring and storing data of biological samples, a method for controlling a biological system and an apparatus for these methods, and uses thereof.

Method for acquiring and storing biometric data, in particular data of biological samples, method for controlling a biological system, apparatus for carrying out the methods, and uses

The present invention relates to a method for acquiring and storing biometric data of any kind, in particular data of biological samples, a method for controlling a biological system and an apparatus for these methods, and uses thereof.

Biological samples produce a multiplicity of electromagnetic signals that can be acquired by means of, for example, an electroencephalogram (EEG), electronystagmography (ENG), an electrocardiogram (ECG) or else, for example, on individual cells by means of patch-clamp methods. In the same way as biological samples produce electromagnetic signals, biological samples, and hence also biological processes, can be influenced by means of electromagnetic signals. All of the aforementioned techniques can also be used to influence or, for example, stimulate biological samples. This is known for the influence of electromagnetic fluxes or electromagnetic fields on nerve cells/neurons, e.g., of the central nervous system, which is also used, for example, for techniques such as biophoton spectral analysis, biological resonance techniques and the like. Biological samples can be influenced not only by electromagnetic fields but also by magnetic fields or in other ways.

Expressed differently, the acquiring of electromagnetic signals from biological samples and influencing biological samples by electromagnetic or other signals is conventionally used in many fields and, in particular, in the field of medicine.

Furthermore, a multiplicity of further information items and data are obtained in the prior art about biological samples and biological systems, for example image data, biochemical data such as laboratory values or histology findings, for example in the form of text, images, videos or the like.

However, these data are often analogue information items which, these days, are subsequently converted into digital data for storage purposes in all of the aforementioned methods. However, very large amounts of data arise in the process; these have to be stored in a suitable manner.

Particularly if progression data or comparison data are recorded, for example prior to and after a medical treatment, prior to and after the occurrence of symptoms and the like, the arising large amounts of data can only be processed with difficulties. Here, in particular, storing has to be implemented predominantly, but not exclusively, in the medical field, which is sensitive in this respect, so that only authorized persons, such as, for example, medical staff, research staff, system administrators, etc., can access the data. However, securing access and confidentiality of the information is linked with large additional outlay in the case of the arising large amounts of data. Similarly, the collected and stored data must also be protected from manipulation. Additional processes should also be planned to this end.

In view of these challenges, it is an object of the present invention to provide a method and an apparatus for acquiring biometric data of any kind, in particular data of biological samples, in which the large amounts of arising data can be processed with secured access and secured integrity of the data.

According to the method according to the invention, biometric data, in particular of biological samples or systems, for example electromagnetic signals emitted by a biological sample, are acquired as analogue or digital electric signals or data. By way of example, this can be carried out like in conventional methods of this type by means of EEG, ECG, ENG, patch clamp by means of photography or video recordings or the like and, as such, it is not a novel method step.

In a further step, the data acquired in this way, provided this relates to analogue data, are converted into digital data.

Conventionally, the conversion is implemented in analogue-to-digital converters or the like and, as such, it is also sufficiently well known from the prior art.

Reference is largely made to “biological samples” above, below and in the claims, with this term also intending to include entire biological systems, up to whole organisms or living beings. However, if necessary, the human and/or animal body as such should be excluded from this if need be.

What is decisive about the method according to the invention for acquiring biometric data, in particular data of biological samples is that the digital data are stored in unmodified fashion or after modification in a blockchain together with a timestamp.

The integrity of the data is ensured by storing the digital data in a blockchain since a blockchain is inherently unforgeable. Furthermore, what is essential here is that a timestamp is additionally stored for the biometric data, in particular biological data, in the blockchain. Said timestamp allows unique labelling of the state of the biological sample acquired with the digital data at the time or during the time period specified by the timestamp and it makes said data accessible to subsequent comparisons.

Here, the timestamp can advantageously be a time or time period at which, or during which, the aforementioned biometric data were acquired, e. g. data were acquired from biological samples, and/or the digital data are/were stored.

With the aid of this timestamp, it is possible to record data about the biological sample including biometric data, said data being assigned to different times or time periods, and thus carry out a comparison between data from different times/time periods. By way of such comparison, it is possible, for example, to determine interim changes, for example on account of a disease or an account of a therapy.

The digital data established from the acquired data can be modified prior to storage or after storage. The modified data or influences, based on these modified data, produced thus can subsequently be reapplied to a biological system, e. g. the same biological sample, in a further configuration of the method according to the invention, for example into order to control the latter therewith (“bio-steering”). Possible methods for influencing a biological sample in accordance with the modified data are sufficiently well described in the prior art and are generally known methods, such as, for example, infusion techniques, gene manipulation techniques, instances of irradiation or another treatment with chemicals. These generally known methods are described in detail, inter alia in the following textbooks:

Lehrbuch der molekularen Zellbiologie [Textbook of molecular cell biology], 4th edition (2012), WILEY-VCH Verlag, Weinheim, Bruce Alberts et al., Translation of chapter 10.1 Manipulation und Analysen von DNA-Molekiilen [Manipulation and analyses of DNA molecules], p. 351ff

“Micromachines as Tools for Nanotechnology”, 2003, H. Fujita, University of Tokyo Springer Verlag, Berlin; Chapter 3.1.1ff

“Molekulare Biotechnologie” [Molecular biotechnology] Michael Wink, Verlag Wiley-Blackwell, 2011, 2nd edition, chapter 15.1/p. 159ff

“Bioinformatik Interaktiv—Algorithmen und Praxis” [Interactive bioinformatics—algorithms and practice] (2010); Merkel, R.; Waack, S., Verlag Wiley-VCH, ISBN 978-3-527-32594-8

By way of example, leucocytes can be modified, for example gene manipulated, by irradiation with electromagnetic signals (x-rays, UV light, etc.); see for example:

“Leukemic cell intracellular responses to nanosecond electric fields” Nianyong Chen, K. H. Schoenbach, Juergen F. Kolb, R. James Swanson, Allen L. Garner, Jing Yang, Ravindra P. Joshi, Stephen J. Beebe, in: Biochemical and Biophysical Research Communications, Volume 317, Issue 2, 30 Apr. 2004, Pages 421-427;

“DNA-fragmentation in human fibroblasts under extremely low frequency electromagnetic field exposure” Focke, F., Schuermann, D., Kuster, N., Schär, P, in: Mutation Research—Fundamental and Molecular Mechanisms of Mutagenesis; 2010; 683 (1-2): 74-83, or

“Analysis of DNA fragmentation in mouse embryos exposed to an extremely low-frequency electromagnetic field”, Bohani, N., Rajaei, F., Salehi, Z., Javadi, A., Electromagnetic Biol Med 2011; 30 (4): 246-252

In the field of autologous blood therapy, too, irradiation for changing the respective blood sample is known. Further methods, which are already known sufficiently well, relate to the use of intravenous immunoglobulins (IVIGs). By way of example, gene manipulations can be carried out by means of electromagnetic fields or other extracellular stimuli; see, for example

“Nanosecond Pulsed Electric Field Stimulation of Reactive Oxygen Species in Human Pancreatic Cancer Cells is Ca2+-Dependent”, Richard Nuccitelli, Kaying Lui, Mark Kreis, Brian Athos, Pamela Nuccitelli, in: Biochem Biophys Res Commun; 2013; Jun. 14; 435 (4): 580-585

By way of example, irradiation with x-rays, UV light, blue light, red light and the like are suitable as irradiation techniques; see for example
i) by UV irradiation:

“Optofluidic Devices for Cell, Microparticle and Nanoparticle Manipulation” Aaron Takami Ohta, EECS department, University of California Berkley; UCB/EECS-2008-148, 2008; p. 76ff

“UVB and Gamma induced DNA fragmentation in resting T-Cells”; Allen Press In: Experimental Hematology; vol. 20/issue 1; 1992; p. 81ff
ii) by chemical substances:

“Differential DNA fragmentation patterns induced by fluoropyrimidines” Christine Elizabeth Canman; PhD thesis; University of Michigan; 1993; p. 8ff
iii) or else by electroporation by means of strong electric fields:

E. Neumann, M. Schaefer-Ridder, Y. Wang, P.H. Hofschneider: “Gene transfer into mouse lyoma cells by electroporation in high electric fields.” EMBO Journal, volume 1(7), 1982, p. 841-845. PMID 6329708; PMC 553119.

C.N. Haas, D. Aturaliye: “Semi-quantitative characterization of electroporation-assisted disinfection processes for inactivation of Giardia and Cryptosporidium”. Journal of Applied Microbiology. Volume 86, number 6, Jun. 1999, p. 899-905, ISSN 1364-5072. PMID 10389240.

C. Liu, X. Xie, W. Zhao, N. Liu, P.A. Maraccini, L. M. Sassoubre, A. B. Boehm, Y. Cui: “Conducting nanosponge electroporation for affordable and high-efficiency disinfection of bacteria and viruses in water”. Nano letters. Volume 13, number 9, Sep. 2013, p. 4288-4293, ISSN 1530-6992.

L. A. Johnson, B. Heemskerk, D. J. Powell Jr, C. J. Cohen, R. A. Morgan, M. E. Dudley, P. F. Robbins, S. A. Rosenberg: “Gene transfer of tumor-reactive TCR confers both high avidity and tumor reactivity to nonreactive peripheral blood mononuclear cells and tumor-infiltrating lymphocytes”. J. Immunol. 177(9), 1 Nov. 2006, p. 6548-6559.

C. Krug, M. Wiesinger, H. Abken, B. Schuler-Thurner, G. Schuler, J. Dörrie, N. Schaft: “A GMP-compliant protocol to expand and transfect cancer patient T cells with mRNA encoding a tumor-specific chimeric antigen receptor”. Cancer Immunol Immunother. 63(10), October 2014, p. 999-1008.

J. A. Kyte, G. Gaudernack: “Immuno-gene therapy of cancer with tumour-mRNA transfected dendritic cells”. Cancer Immunol Immunother. 55(11), November 2006, p. 1432-1442. Epub 2006 Apr. 13.

S. Wilgenhof, J. Corthals, A. M. Van Nuffel, D. Benteyn, C. Heirman, A. Bonehill, K. Thielemans, B. Neyns: “Long-term clinical outcome of melanoma patients treated with messenger RNA-electroporated dendritic cell therapy following complete resection of metastases”. Cancer Immunol Immunother. 64(3), March 2015, p. 381-388.

Michael K. Stehling, Enric Günther, Boris Rubinsky: Mit Stromstöβen gegen Krebs [Fighting cancer with current surges]. Spektrum der Wissenschaft. April 2014, p. 40. DIN IEC/TS 60479-1 (VDE V 0140-479-1):2007-05 “Wirkungen des elektrischen Stromes auf Menschen and Nutztiere—Teil 1: Allgemeine Aspekte” [Effect of the electric current in humans and livestock—part 1: general aspects], chapter 5.6, p. 26.

In all these methods, different means are used to influence the biological sample, for example infusion, irradiation and the like. The present invention uses such known methods, with influencing according to the invention being implemented depending on the aforementioned digital data, either in unmodified form or post modification.

Particularly in order to acquire the influences of disease or the influences of effects such as therapeutic effects, for example, on a biological sample, it is possible to carry out temporally successive acquisitions of biometric data of the same sample and establish the effect of the disease or of the treatment/the influence therefrom. This also renders it possible to modify data in such a way that they are cleansed from influences that are obviously ascribed to the disease or the other influencing before the use to act on the biological sample as described above.

In order to ensure data security, the data can optionally also be encrypted, for example by using a symmetric key or by using an asymmetric key. Storing data encrypted in this way in a blockchain provides the greatest possible integrity of the data and the greatest possible access control to this data. This is because access to the data is not possible without knowledge of the corresponding key. Therefore, the access to the data, which may readily contain sensitive data, can be strictly regulated and controlled by providing the key to selected persons.

By way of example, a file that was created with the aid of the DNA of the respective human/the respective patient whose sample should provide the data can be used as a private key for the signature in an asymmetric encryption method. This allows a generation of an individual key for the encryption since the DNA of each human is individual. The DNA or the file with information about the DNA of the respective human created herefrom can be used to determine the key in a symmetric or an asymmetric encryption method.

It is advantageous if the file with the information items about the DNA itself is used as a key.

The use of a blockchain for storing the acquired data, which have been converted into digital data, is furthermore advantageous in that the length of a blockchain is not restricted as a matter of principle. Therefore, it is always possible to write additional data into the blockchain and thus constantly expand the number of data records stored there. As a result of the hash value thereof once again being calculated and stored after the respective modification of the blockchain, the overall integrity of the data, including the previously stored data and the most recently added data, is always ensured.

The modulated data, too, can be stored in the blockchain, for example together with a time stamp indicating the time at which the data for establishing an instance of influencing of the biological sample were used or at a time at which the biological sample was influenced on the basis of these modified data, as described above.

According to the invention, the method or an apparatus used to this end can (but need not) be used to carry out a diagnosis and/or else a control (within the meaning of biotechnological steering) such as a therapy, for example, on biological samples such as whole organisms like plants, animals, living cells, organs and the like. To the extent that the biological sample is a living human or animal body, the diagnosis and the therapy is implemented within the respective legal scope. However, the diagnosis and the therapy of a biological sample that is a human body or an animal body or a non-severed constituent thereof may also be excluded from the method according to the invention.

The apparatus according to the invention, as claimed in the claims, now has a detection unit for acquiring biometric data, e. g. data of a biological sample, as analogue data or digital data. Furthermore, it optionally has a conversion unit for converting the analogue data into digital data. Furthermore, it has a memory unit for storing the digital data in a blockchain together with a timestamp. There can advantageously be a corresponding encryption unit for encrypting the digital data, there can be a modification unit for modifying the data in an inventive manner and there can be an effective unit for exerting an influence on the biological sample depending on the stored data, including or excluding a modification carried out thereon for influencing the biological sample in an inventive manner.

An apparatus according to the invention and a method according to the invention will now be described below on the basis of an example.

In the drawing:

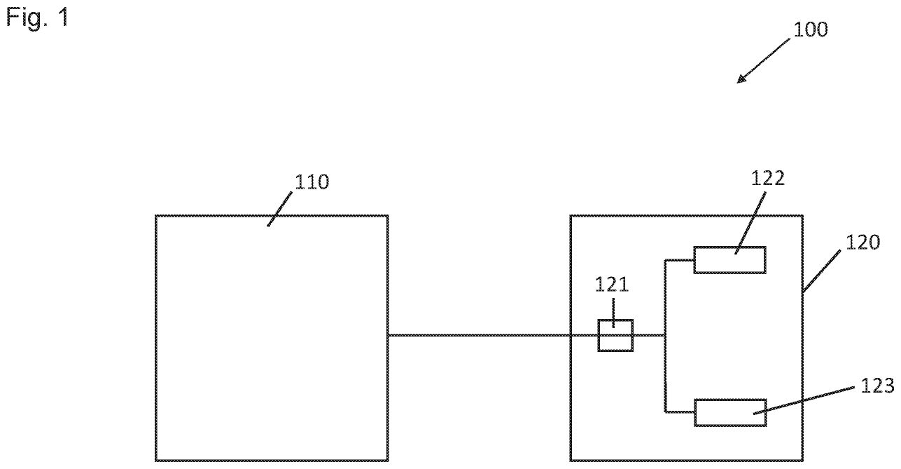

FIG. 1 shows an apparatus according to the invention in individual layers,

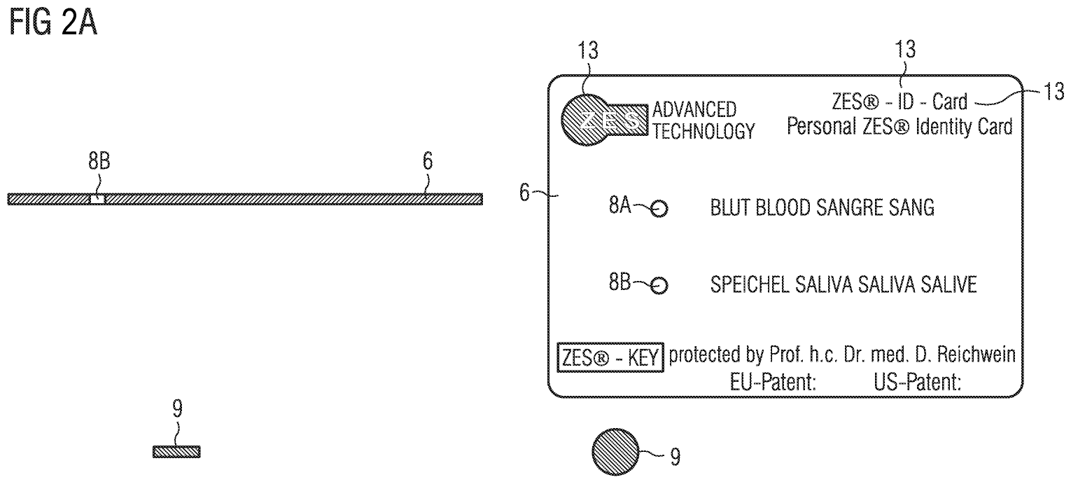

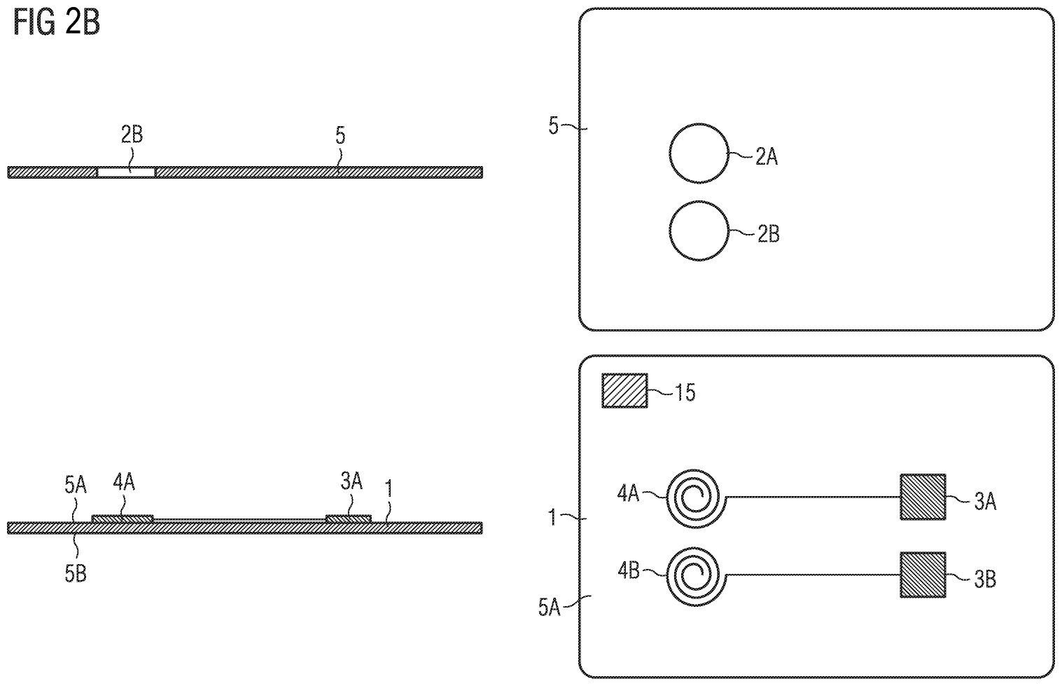

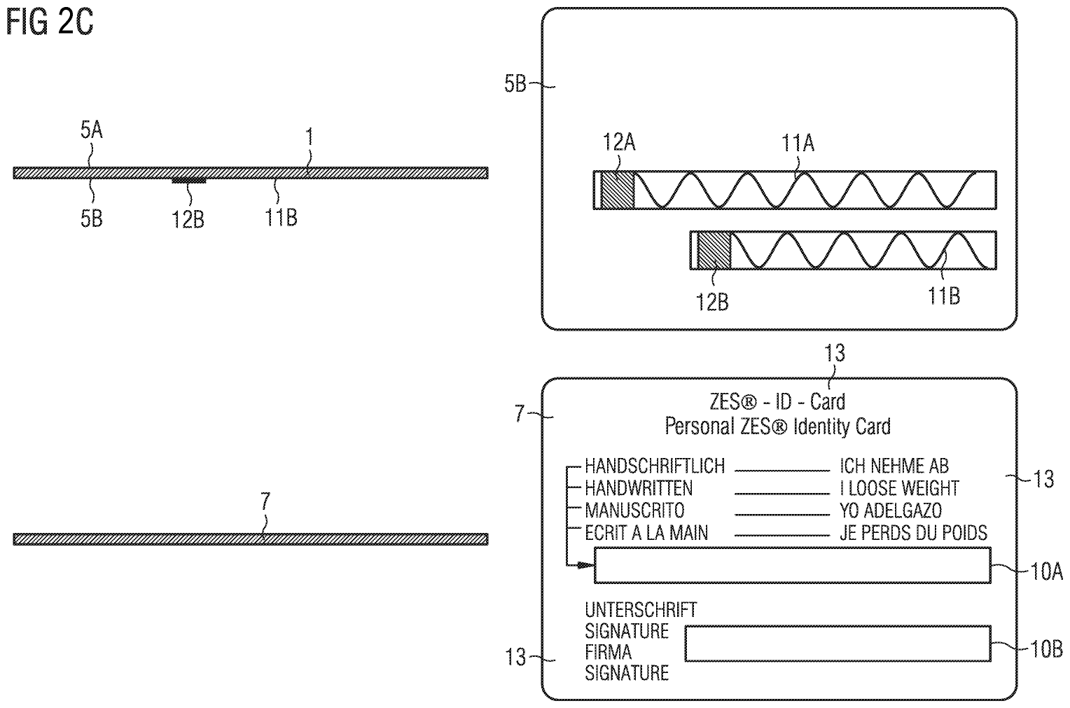

FIGS. 2A, 2B and 2C show a transmitter/receiver apparatus in individual layers; and

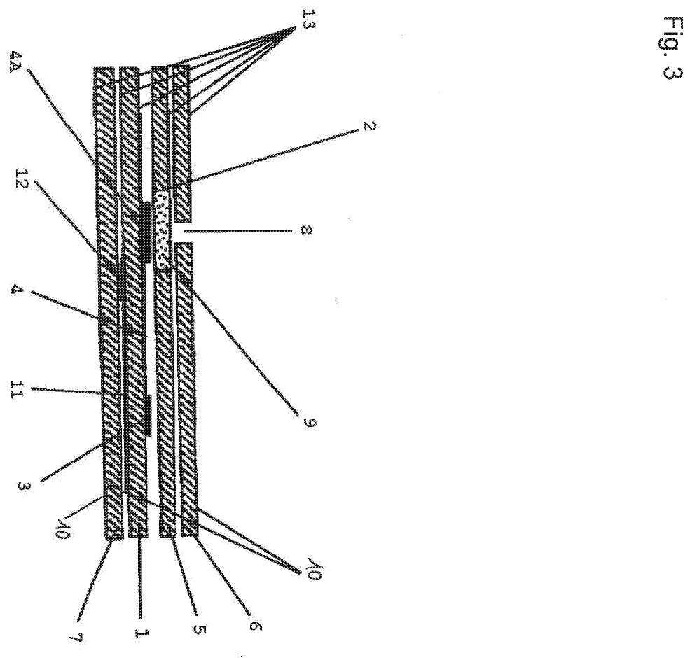

FIG. 3 shows the structure of an apparatus according to the invention.

FIG. 1 shows an apparatus according to the invention for carrying out the method according to the invention. Firstly, it has a transmitter/receiver apparatus 110, which will be described in detail below with reference to FIGS. 2 and 3. This transmitter/receiver apparatus 110 receives electric signals as data, for example electromagnetic waves from a biological sample. In turn, it is also capable of emitting electrical signals, for example electromagnetic waves onto a biological sample. Expressed differently, the transmitter/receiver apparatus 110 is an electromagnetic receiver and/or transmitter that has been provided with a dipole antenna, for example.

Furthermore, the apparatus according to the invention has a processing apparatus 120. The processing apparatus 120 has an input/output unit 121 as well as a memory 122 and a processing unit 123. Input/output unit 121, memory 122 and processing unit 123 are linked to one another, for example by way of a data bus. The input/output unit 121 has an A/D converter for converting the analogue electromagnetic signals that are received and transmitted by the apparatus 110 into digital signals. Furthermore, the input/output unit 121 has a D/A converter for converting the digital signals output by the processing unit 123 or the memory 122 into analogue signals that can then be transmitted to the transmitter/receiver apparatus 110.

The signals converted into digital signals by the A/D converter in the input/output unit 121 are, firstly, written to the memory 122 and, secondly, processed in the processing unit 123.

The data processing, including modifying the data and providing the data with a timestamp, as described in the claims occurs in the processing unit 123. Subsequently, the timestamp-provided data modified thus are written to the memory 122. Instead of the local memory 122, use can also be made of a cloud memory, which is accessed via a public network, for example the Internet.

The modified data can be transmitted to the transmitter/receiver apparatus 110 via the D/A converter in the input/output unit 121 and can be emitted there as an electromagnetic wave by way of the antenna situated therein.

The method according to the invention is carried out by virtue of the transmitter/receiver apparatus 110 being arranged in the vicinity of a biological sample, for example a tissue sample (e.g., a punch biopsy) or a blood sample. The electromagnetic signals emitted by the biological sample are acquired by the transmitter/receiver apparatus 110 and transmitted to the processing apparatus 120. There, they are converted into digital data in the input/output unit 121 by means of an A/D converter, provided with a timestamp by the processing unit 123 and stored in a blockchain or added to an existing blockchain in the memory 122. The data stored thus can be modified, for example by virtue of changes in the digital signals over time being captured and being compensated by suitable processing. The data modified thus can be stored in the memory 122, for example in the same blockchain as the initial data, subsequently be forwarded to the input/output unit 121, converted into analogue electric signals there by means of a D/A converter and transmitted to the transmitter/receiver apparatus 110. The transmitter/receiver apparatus 110 converts the received analogue signal into an electromagnetic wave by means of an antenna, said electromagnetic wave being emitted and being able to be received by the biological sample, which is arranged in the vicinity of the transmitter/receiver apparatus 110.

Storing the data in a blockchain firstly ensures the integrity of the data and secondly records the data history together with the timestamp. This renders it possible to observe the profile of the recorded signals, for example, during an illness or during therapy.

FIG. 2 shows a preferred configuration of the transmitter/receiver apparatus 110 according to the invention, with individual layers being illustrated in their sequence. Individual layers 1, 5, 6, 7 of the apparatus 110 are illustrated in a side view (A) and in a plan view (B). Each layer 1, 5, 6 and 7 has a planar embodiment with the chip card form (L×W×H=approximately 8.6 cm×approximately 5.4 cm×approximately 0.8 cm).

The first layer 1 is configured as a printed circuit board made of epoxy resin with a thickness of 0.37 mm, which, as conductor tracks made of copper, contains two capacitors 3A, 3B on the right-hand side as apparatuses for storing electromagnetic energy and two single conductors 4A, 4B. On one side, the capacitors 3A and 3B are each connected to a first end of the respective single conductor 4A and 4B. The second connector of the capacitors 3A, 3B is free or insulated.

The free second end of the single conductors 4A, 4B has a spiral shape in each case and it is arranged on the left-hand side of the layer 1. Furthermore, the first layer 1 has on its first side a third capacitor 15 as an apparatus for storing electromagnetic energy. A further electric signal can be written into this capacitor. This capacitor is electrically connected to the processing apparatus 120 by a further line that cannot be identified in the figure and it is able to transmit analogue electric signals to said processing apparatus and receive said signals therefrom.

The second layer 5 consists of polyethylene, it has a thickness of 0.20 mm and it is adhesively bonded to the surface (first side) of the first layer 1 that is provided with the conductor tracks 3A, 3B, 4A, 4B. The second layer 5 contains two circular cavities 2A, 2B that pass therethrough, have a diameter of approximately 8.5 mm in the length plane and are arranged exactly over the free spiral-shaped end of the single conductors 4A, 4B. The cavities 2A and 2B are terminated on the lower side by the first layer 1 and are open on their upper side. A self-adhesive pad 9, illustrated separately in FIG. 1, is arranged in the cavities 2A and 2B in each case. The self-adhesive pad 9 consists of a circular, absorbent material and it is characterized by a diameter of 8 mm and a thickness of less than 0.20 mm. By way of example, this pad 9 serves to receive a bodily fluid, as specified above.

A third layer 6, illustrated separately here, is adhesively bonded to the second layer 5, said third layer being configured as a decorative film, consisting of polyethylene with a thickness of 0.15 mm and containing two through-cutouts 8A, 8B in the form of through-holes, said cutouts each being situated exactly in the centre above the cavities 2A and 2B of the second layer 5. The through-holes have a diameter of 2 mm. Consequently, apart from the holes, the two cavities 2A and 2B of the second layer 5 are sealed by the decorative film 6.

On its second side 5B, the first layer 1 has two second arrangements, each with a field, a single conductor and an apparatus for storing electromagnetic energy; i.e., the second side of the first layer 1 contains two capacitors 12A, 12B and two wave-shaped single conductors 11A, 11B in the left-hand part. At their one respective connector, the two capacitors 12A, 12B are each connected to one of the wave-shaped single conductors 11A, 11B. The free end of the single conductors 11A and 11B extends to the right-hand side, up to the vicinity of the edge of the layer 5. The other connector of the capacitors 12A, 12b is free or electrically insulated.

Additionally, a fourth layer 7 is arranged on the second side of the layer 1, said fourth layer being configured as a decorative film, consisting of polyethylene with a thickness of 0.15 mm and having an adhesive print and two labelling fields 10A, 10B. The labelling fields 10A, 10B are each situated exactly above the single conductors 11A and 11B of the second arrangement. Since all four layers are adhesively bonded to one another, this yields an apparatus made of four layers 1, 5, 6 and 7 as a result, i.e., a four-layer chip. Possible locations for pre-printed writing 13 are illustrated in the fourth layer 7.

FIG. 3 describes the structure of the configuration of the apparatus according to the invention as per FIG. 2 in a cross section. The apparatus has a plane embodiment in the chip card form (L×W×H=approximately 8.6 cm×approximately 5.4 cm×approximately 0.8 cm) and has a structure made of the four layers 1, 5, 6, 7. The first layer has a capacitor 3 as an apparatus for storing electromagnetic energy on the right-hand side and, in the centre and up to the left-hand side, it contains a single conductor 4 made of copper wire. At its first connector, the capacitor 3 is connected to one end of the single conductor 4. The second connector of the capacitor 3 is free or electrically insulated. The free end of the single conductor 4 (labelled 4A here) has a spiral-shaped embodiment and is arranged on the left-hand side of the layer 1.

The second layer 5 contains a circular through-cutout 2, which has a diameter of approximately 8.5 mm in the plane of the layer, and is arranged exactly over the spiral-shaped free end 4A of the single conductor 4 in the left-hand part of the layer 5. In the completed apparatus, the through-cutout 2 is terminated by the layer 1 on its lower side (second side) and open on its upper side (first side) and consequently forms a cavity. A self-adhesive pad 9 is arranged in this cavity.

The third layer 6 is adhesively bonded on the second layer, said third layer 6 containing the through-hole 8, which is situated exactly in the centre over the cavity, i.e., over the through-cutout 2 of the second layer 5. The through-hole 8 of the third layer 6 has a diameter of 2 mm in this example. Apart from the through-hole 8 in the third layer 6, the cavity 2 is sealed by the decorative film 6. On its upper side, this decorative film 6 carries various fields 13 with imprints. The cavity 2, the capacitor 3 and the conductor track 4 on the first side of the first layer 1 form a first arrangement.

The further layer 7, which has a printed field 13 and an inscribable labelling field 10 on its outer side, is adhesively bonded to the opposite second side of the layer 1. In the region of the labelling field 10 and lying opposite thereto on the second side of the second layer 5, a wave-shaped single conductor 11 is arranged as a conductor track. The one end of the single conductor 11 is connected to a capacitor 12 embodied as a conductor track in the region of the left-hand end of the labelling field 10 and opposite thereto on the second side of the second layer 5. The other end of the conductor 11 is free and extends in wave-shaped fashion from the capacitor 12 to the vicinity of the right outer edge of the layer 5. The field 10, the capacitor 12 and the conductor 11 form a second arrangement.

Since all four layers 1, 5, 6 and 7 are adhesively bonded to one another or welded to one another, an apparatus made of four layers arises, i.e., a four-layer chip card.

In general, at least one second arrangement can be provided on the second side of the substrate in the transmitter/receiver apparatus according to the invention, said at least one second arrangement having at least one field (10), at least one second single conductor (11) and at least one second apparatus for storing electromagnetic energy (12), with one end of the second single conductor (11) being connected to the second apparatus for storing electromagnetic energy (12) and the other end being arranged in, on or adjacent to the field (10) as a free end.

In the apparatus according to the invention, the at least one second arrangement can be arranged in, on or adjacent to the second side of the first layer.

In the transmitter/receiver apparatus according to the invention, a fourth layer (7) can be arranged on the second side of the first layer, said fourth layer having at least one field (10) or at least one through-cutout in the form of a field (10) and preferably being designed as a self-adhesive decorative film.

In the transmitter/receiver apparatus according to the invention, the fourth layer (7) can contain or consist of plastic, preferably polycarbonate, polyethylene or polyvinyl chloride.

In the transmitter/receiver apparatus according to the invention, the field (10) or the through-cutout in the form of a field (10) can be rectangular, preferably have the dimensions length×width of 2-7 cm×0.5cm, particularly preferably 4-6 cm×0.8-1.5 cm, or be circular or oval.

In the transmitter/receiver apparatus according to the invention, the at least one field (10) or the at least one cutout in the form of a field (10) can have a writable material.

In the transmitter/receiver apparatus according to the invention, the writable material can be embodied in such a way that it absorbs and/or adsorbs solid and/or liquid writing material.

In the transmitter/receiver apparatus according to the invention, the at least two layers (1, 5, 6, 7) of the substrate can be largely round, oval or rectangular.

In the apparatus according to the invention, the at least two layers (1, 5, 6, 7) of the substrate can be embodied in card form, preferably having length×width×height dimensions of 2-14 cm×2-10 cm×0.05-0.60 mm, particularly preferably of 8-9 cm×5-6 cm×0.15-0.40 mm.

In the transmitter/receiver apparatus according to the invention, a third apparatus for storing electromagnetic energy can be arranged on, in or adjacent to the substrate.

In the transmitter/receiver apparatus according to the invention, at least one, some or all of the apparatuses for storing electromagnetic energy (3, 12) can be a capacitor or can have a capacitor.

In the transmitter/receiver apparatus according to the invention, the apparatus on the first side can have one, two or three first arrangements.

In the transmitter/receiver apparatus according to the invention, the apparatus on the second side can have one, two or three second arrangements.

In the transmitter/receiver apparatus according to the invention, the free end of the at least one single conductor (4) on the first side can have a spiral-shaped embodiment.

In the transmitter/receiver apparatus according to the invention, the free end of the at least one single conductor (11) on the second side can have a wave-shaped embodiment.

In the transmitter/receiver apparatus according to the invention, the free end of the at least one single conductor (4) on the first side can be worked into the through-opening in the second layer (2), preferably in such a way that the single conductor (4) contacts the first and/or second layer (1, 5) at least in regions in the region of the through-opening (2).

In the transmitter/receiver apparatus according to the invention, the free end of the at least one single conductor (11) on the second side is worked into the field (10) or into the cutout in the form of a field (10), preferably in such a way that the single conductor (11) contacts the first layer (1), at least in regions.

In the transmitter/receiver apparatus according to the invention, the single conductor on the first side (4) can contact the first, second and/or third layer (1, 5, 6) on less than half or on half of its circumference and/or the single conductor on the second side (11) can contact the first layer and/or fourth layer (1, 7) on less than half or on half of its circumference.

In the transmitter/receiver apparatus according to the invention, at least one of the single conductors (4, 11) can contain or consist of substantially electrically conductive material, preferably ferromagnetic material; and/or, it can be coated, at least in regions, with at least one noble metal, preferably gold.

1. A method for acquiring and storing data of biological samples, including the following steps:
acquiring the data, characterized in that the data are stored as digital data in unmodified fashion or after modification in a blockchain together with a timestamp.

2. The method according to claim 1, characterized in that the timestamp specifies the time or time period at which or during which the electromagnetic signals are acquired or specifies the time or time period at which the digital data are stored.

3. The method according to claim 1, characterized in that the data of the biological samples are biophysical and/or biochemical data, which were acquired from the biological sample.

4. The method according to claim 1, characterized in that the data of the biological samples are acquired by measurement methods, including acquiring electromagnetic signals emitted by a biological sample, as analogue or digital electric signals, by electron tomography, electrocardiography, electronystagmography, potential measurements on cell membranes, or acquired by imaging methods, including photography of image files of video files, or by other methods, including medical history or tissue assessments.

5. The method for influencing and/or controlling a biological system, with the exception of a human body, including the following steps:
carrying out an acquisition and storage of data according to claim 1, modifying the captured digital data for the purposes of producing modified data before or after the aforementioned storage and
controlling the biological system depending on the modified data.

6. The method according to claim 1, characterized in that the digital data have first digital data, which were acquired as electromagnetic signals or stored as digital signals at a first time, at which the biological sample was in a first state, and second digital data, which were acquired as electromagnetic signals or stored as digital data at a second time, at which the biological sample was in a second state, and
the first digital data are compared to the second digital data and the second digital data are modified according to the result of the comparison.

7. The method according to claim 5, characterized in that the digital data are encrypted in a blockchain prior to storage, including using a symmetric or asymmetric encryption method.

8. The method according to claim 7, characterized in that the data are modified or modulated prior to and/or after encryption and/or prior to and/or after storage.

9. The method according to claim 8, characterized in that the control is carried out by infusion techniques, irradiation techniques or other treatment techniques.

10. The method according to claim 4, characterized in that the conversion of the analogue data into digital data is implemented by an analogue-to-digital converter.

11. The method according to claim 5, characterized in that storage of the digital data in a blockchain is implemented in a memory, including a RAM or a ROM.

12. An apparatus for carrying out the method according to claim 1, comprising
a detection unit for acquiring data, including data emitted by a biological sample, as analogue or digital electric signals,
a conversion unit for converting the analogue electric signals into digital data, and a memory unit for storing the digital data in a blockchain together with a timestamp.

13. The apparatus according to claim 12, characterized by an encryption unit for encrypting the digital data prior to storage in a blockchain, including a symmetric or asymmetric encryption method.

14. The apparatus according to claim 12, characterized by a modification unit for modifying or modulating the data prior to and/or after encryption and/or prior to and/or after storage.

15. The apparatus according to claim 12, characterized by an effector unit for exerting an influence on the biological sample depending on the stored data.

16. The apparatus according to claim 15, characterized in that the effector unit is or contains an infusion system or an irradiation system.

17. The apparatus according to claim 12, characterized in that the detection unit is an EEG appliance or an ENG appliance.

18. The apparatus according to claim 12, characterized in that the conversion unit is an analogue-to-digital converter.

19. The apparatus according to claim 12, characterized in that the memory unit is or contains a RAM or a ROM.

20. Use of the method according to claim 1 for diagnosing or controlling biological samples including living cells, organs, organisms, and living beings with the exclusion of methods on the human body or methods on the animal body.
